(19) United States
(12) Patent Application Publication
(10) Pub. No.: US 2021/0104319 A1
(43) Pub. Date: Apr. 8, 2021

(54) Medical Environment Monitoring Systems

(71) Applicant: Augusto De Melo, Brasilia (BR)

(72) Inventor: Augusto De Melo, Brasilia (BR)

(21) Appl. No.: 17/064,497

(22) Filed: Oct. 6, 2020

Related U.S. Application Data
(60) Provisional application No. 62/911,517, filed on Oct. 7, 2019.

(51) Int. Cl. G16H 40/67; G16H 40/20

(52) U.S. Cl. CPC G16H 40/67; G16H 40/20

(57) ABSTRACT

A medical environment monitoring system suitable for use for augmenting medical monitoring equipment for patients is provided. The system includes a handheld device configured to monitor environmental conditions. The handheld device includes at least one temperature sensor. The handheld device also includes at least one humidity sensor. The handheld device further includes at least one chronometer. The chronometer structured to measure elapsed time. The handheld device is contoured, shaped, and sized to occupy a cavity in the medical monitoring equipment such that when the handheld device is stowed onto the monitoring equipment and occupies the cavity, the cavity is not visible by plain sight. The handheld device mates and connects with the medical monitoring equipment in a releasable fashion. Additionally, the handheld device can be a standalone device.

### RELATED APPLICATIONS

This application claims priority to U.S. Provisional Application No. 62/911,517 filed Oct. 7, 2019. The entire contents of the above application are hereby incorporated by reference as though fully set forth herein.

### FIELD OF THE INVENTION

The present invention relates generally to Environment Monitoring Systems, and more particularly, to the Medical Environment Monitoring System, which utilize add-on and/or standalone portable devices to improve the accuracy in measuring a patient's vital signs, monitoring critical medical procedures, in alerting when to provide critical lifesaving processes, and in providing aesthetic cost effective solutions to manufacturers producing equipment with empty slots.

### BACKGROUND OF THE INVENTION

Many methods and systems have been used unsuccessfully attempting to incorporate add-on medical devices to existing medical environment monitoring equipment in order to improve the capabilities available to medical professionals in an efficient and cost-effective manner. Several devices and methods have been created attempting unsuccessfully to solve the problem of medical environment monitoring equipment being deployed with an existing cavity or lack of monitoring modules. These cavities are often perceived by medical professionals and patients as incomplete or defective devices. Previous unsuccessful attempts to address medical vital sign monitoring equipment being sold with a cavity resulted in expensive manufacturing costs and made these efforts non-cost effective. Further, existing monitoring equipment often lack portable devices which can attach to and/or communicate with the monitoring equipment to provide medical environment monitoring. For example, monitoring equipment do not have the ability of measuring elapsed time, temperature and/or humidity at the point of medical care delivery at a patient's bedside, operating rooms, and intensive care units.

Accordingly, there is an established need for Medical Environment Monitoring System which solve at least one of the aforementioned problems. Further, there is an established need for Medical Environment Monitoring System which can be used as add-on and/or standalone devices wherein the devices can measure and monitor key medical parameters of a patient, can allow a medical professional to utilize the devices in a portable manner to interact in real-time with another medical piece of equipment, and provide cost effective solutions for manufacturers delivering equipment with empty slots.

### SUMMARY OF THE INVENTION

The present invention is directed to innovative Medical Environment Monitoring System which can be utilized as standalone and/or add-on devices to another medical environment monitoring piece of equipment. The devices can provide improved functionality to existing equipment by providing medical professionals the ability to measure and monitor in real-time environmental and medical parameters. Additionally, these innovative Medical Environment Monitoring System can be used on a plurality of types and/or pieces of medical environment monitoring equipment that are sent to end-users with an existing empty slot. Further, the innovations can provide for remote display screens and input capability directly on portable devices and can include additional sensors for augmenting existing medical environment monitoring equipment capabilities. Further still, the innovations can provide for add-on devices which fit in empty slots and/or cavities of other medical equipment thereby providing a cost-effective solution for equipment manufacturers to avoid the perception that their equipment, when delivered with empty slots, are not incomplete or defective.

The sensors can measure parameters such as, but are not limited to, environmental temperature and humidity, time, and/or elapsed time for procedures.

In an aspect, a medical environment monitoring system suitable for use for augmenting medical monitoring equipment for patients is provided. The system includes a handheld device configured to monitor environmental conditions. The handheld device includes at least one temperature sensor. The handheld device also includes at least one humidity sensor. The handheld device further includes at least one chronometer. The chronometer structured to measure elapsed time. The handheld device is contoured, shaped, and sized to occupy a cavity in the medical monitoring equipment such that when the handheld device is stowed onto the monitoring equipment and occupies the cavity, the cavity is not visible by plain sight. The handheld device mates and connects with the medical monitoring equipment in a releasable fashion.

In another aspect, a medical environment monitoring system suitable for use for augmenting medical monitoring equipment for patients is provided. The system includes a handheld device configured to monitor environmental conditions. The handheld device includes at least one temperature sensor. The handheld device also includes at least one humidity sensor. The handheld device further includes at least one chronometer, the chronometer structured to measure elapsed time. Additionally, the handheld device is a standalone device.

According to an aspect of the present invention, a system suitable for use as a standalone and/or add-on medical vital signs system is provided. Further, the medical environment monitoring system may use the electrical power from other pieces of medical equipment where it is attached on, or have its own power supply built in.

According to another aspect of the present invention, the system can also include contoured shapes. The shapes can be contoured to allow the system to fit into cavities or empty slots of other medical equipment. Further, the shapes can include ovals, rectangles, squares, trapezoids, circles, ellipses, squares, and/or curved shapes existing in the x, y, and/or z axes.

According to yet another aspect, the system can include a touch sensitive, or non-touch sensitive, LCD screen. The touch sensitive LCD screen can be configured to display data and/or provide a data input mechanism.

In an embodiment, the stand alone system can include an articulating arm. The articulating arm can be configured to allow attachment of embodiments of the medical environment monitoring system to an existing piece of medical equipment and/or to provide articulation of 360 degrees in the x, y and z axes. The system can include a VESA connection.

In another embodiment, the system can also include solar panels. The solar panels can be configured to provide a power source to a medical environment monitoring system and/or medical equipment, where solar light and/or a well illuminated environment are available

In yet another embodiment, the system can be installed to different types of devices including but not limited to an anesthesia machine, Intensive Care Unit (ICU) monitors, incubators, neo-natal care monitors, surgical suite medical equipment control panel, and/or a patient monitoring and connectivity platform.

In an aspect the system can include a fitted cover. The cover can include materials such as but not limited to plastic, rubber, composites, wood, metal, fiberglass, and/or glass. The materials can be arranged to optimize aesthetic attraction, minimize adverse chemical reactions between the cover and a host unit, minimize any potential galvanic corrosion, and/or configured for use in sterile areas.

In another aspect the system can also include a vital sign monitor. Additionally, the system can be installed with different types of devices where the use of an articulated arm is possible.

In yet another aspect the system can include connections and devices to interface with connectivity platforms.

In an embodiment, the system can include a touch screen. Additionally, the system can include at least one touchscreen.

In another embodiment, the system can also include portable monitoring devices wherein the portable monitoring devices can be stored in a cavity of a piece of medical equipment.

In yet another embodiment, the system can include standalone and/or add-on devices wherein the devices include tablets, smartphones, and/or mobile devices configured for real-time data gathering and allow medical professionals to diagnose a patient's medical condition with algorithms, software, and/or other medical environment monitoring equipment.

In an aspect, the system can include a cover. Additionally, the system can include sensors for measuring temperature, humidity, time and elapsed time. Further, the system can be a standalone system.

In another aspect, the innovative Medical Environment Monitoring System, can include medical vital sign parameters and can be used as an add-on and/or standalone system.

Additionally, these innovative Medical Environment Monitoring System can be used on a plurality of types and/or pieces of medical vital sign monitoring equipment that are sent to end-users with an existing slot, but not restricted to it since the Medical Environment Monitoring System can be also standalone, without the need of an empty slot.

These and other objects, features, and advantages of the present invention will become more apparent from the attached drawings and the detailed description of the preferred embodiments, which follow. It is understood, that the drawings are designed for the purposes of illustration and not as a definition of the limits of the embodiments of the present invention. It should be further understood that the drawings are not necessarily drawn to scale and are merely intended to conceptually illustrate the methods and systems described herein.

### BRIEF DESCRIPTION OF THE DRAWINGS

The preferred embodiments of the invention will hereinafter be described in conjunction with the appended drawings provided to illustrate and not to limit the invention, where like designations denote like elements, and in which:

FIG. 1 presents a front view of a medical environment monitoring system with a screen, in accordance with an embodiment of the present invention;

FIG. 2 shows a top perspective view of an embodiment of the present invention with a screen;

FIG. 3 discloses a side view of an embodiment of the system;

FIG. 4 displays a bottom view of an aspect of the present invention;

FIG. 5 illustrates a rear view of a system in accordance with an embodiment of the present invention;

FIG. 6 displays a front view of an embodiment of a medical environment monitoring system alongside an interface device; and

FIG. 7 shows a front view of an embodiment of the present invention embedded onto an interface device.

Like reference numerals refer to like parts throughout the several views of the drawings.

### DETAILED DESCRIPTION

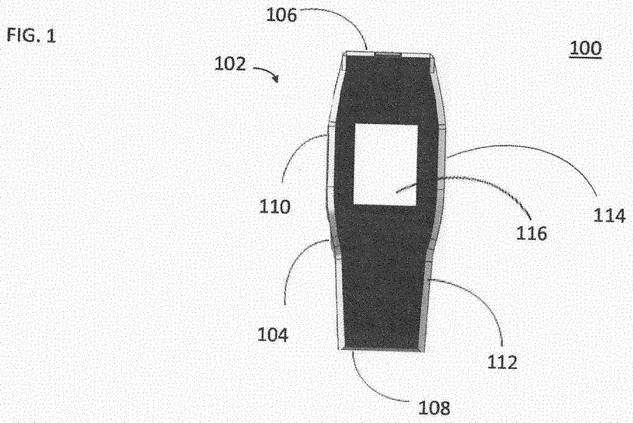

The following detailed description is exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All the implementations described below are exemplary implementations provided to enable persons skilled in the art to make or use the embodiments of the disclosure and are not intended to limit the scope of the disclosure, which is defined by the claims. For purposes of description herein, the terms “upper”, “lower”, “left”, “rear”, “right”, “front”, “vertical”, “horizontal”, and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Hence, specific dimensions and other physical characteristics relating to the embodiments disclosed herein are not to be considered as limiting, unless the claims expressly state otherwise.

Shown throughout the figures, embodiments of the present invention are directed towards methods and systems for Medical Environment Monitoring System with devices and sensors. These devices and sensors can function in concert and be configured as an integrated medical environment monitoring system.

Referring initially to FIG. 1, a medical environment monitoring system 100 is illustrated with an embodiment of the present invention. As seen in FIG. 1, a medical environment monitoring system 100 can include a portable handheld device 102. The device 102 can include a protective case 104. The case 104 can include a top 106, a bottom 108, a left side 110, and a right side 112. The device 102 can include an opening 114 wherein the opening 114 can be configured to house a screen 116. In embodiments not shown, the screen 116 can include, but is not limited to, a liquid crystal display (LCD), Light emitting Diode display (LED), Organic light emitting diode display (OLED), and/or Quantum dot light emitting diode display (QLED).

In embodiments not shown, the screen 116 can include a touch screen. Additionally, the system 100 can include temperature, humidity, date, time, and elapsed time sensors. The screen 116 can be configured to display data from the sensors. Further, the screen 116 can be configured to include a touch screen, wherein the touch screen allows for user input, to toggle among a plurality of displays, toggle among a plurality of parameters, and/or to start and/or stop a timing function emulating a stopwatch. The system can also include a data processor and/or a computer. The system can include a memory storage device.

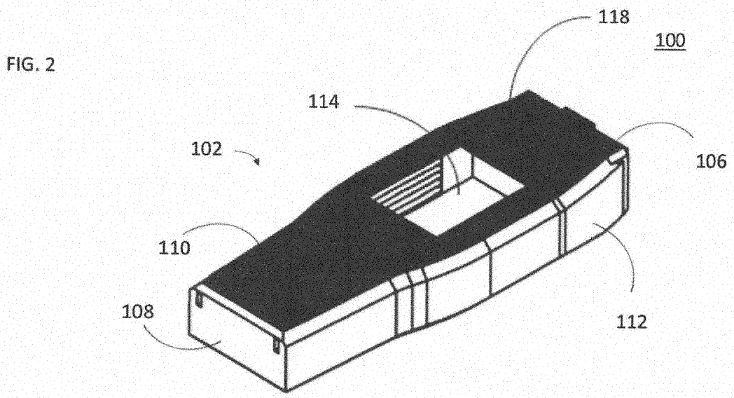

Turning to FIG. 2, a top perspective view of an embodiment of the system is illustrated. The system 100 can include a portable device 102. The device can include a front side 118. As best seen in FIG. 2 the portable device 102 can house other components within a cavity of a body of the device 102.

In embodiments, the system 100 can include an opening 114 in the portable device 102. In embodiments not shown, the opening 114 can be configured to house sensors, electronics, data processors and/or computers, screens, wireless and/or hard wire communication mechanisms, and/or power supply transmission capability apparatus. The system 100 can be configured such that the portable device 102 can measure environmental parameters adjacent to a patient offering an accurate assessment of the patient's environment. The system 100 can be configured to measure and display temperature, humidity, and elapsed time measurements commensurate with a stop watch.

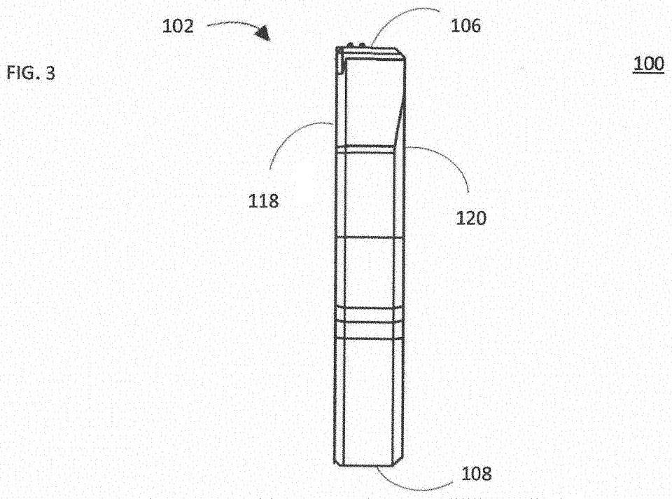

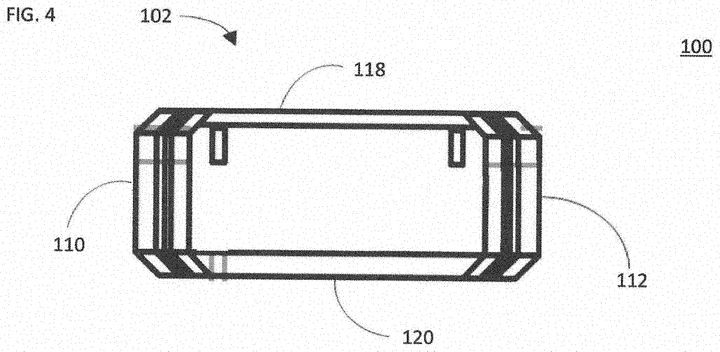

As shown in FIGS. 3 and 4, a side view and a bottom view of the system 100 illustrates a back side 120 of the portable device 102.

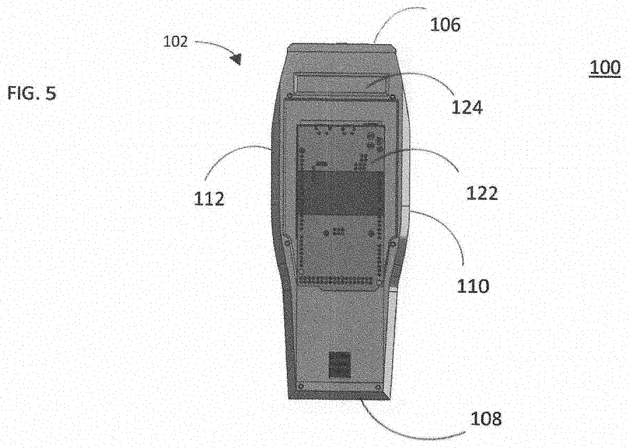

Referencing FIG. 5, a rear view of the portable device 102 is seen. The portable device can include a backside component cavity 122 and a backside connection cavity 124. In some embodiments not shown the component cavity 122 can house sensors, electronics, data processors and/or computers, screens, wireless and/or hard wire communication mechanisms, and/or power supply transmission capability apparatus. Further, the connection cavity 124 can be configured to house connection components configured to transmit and/or receive data and to transmit and/or receive power.

The system 100 can be configured to accurately measure, monitor, and transmit localized patient environment data. The system 100 can be configured to provide the data in real-time to medical care professionals in order to optimize medical treatment.

The system 100 can be configured to utilize wireless communication technology such as but not limited to wi-fi and Bluetooth.

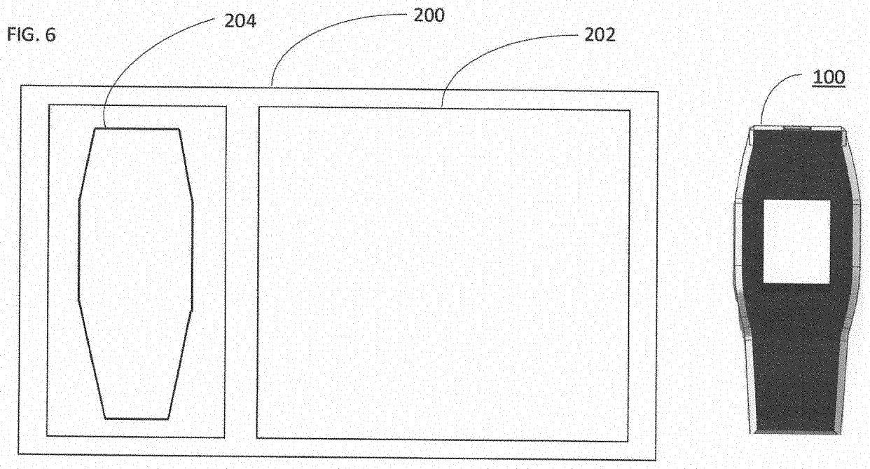

Turning to FIG. 6 a medical monitoring equipment user interface device 200 alongside a standalone medical environment monitoring system 100 are displayed. In embodiments, the user interface device 200 can include communications screens 202 wherein users can interact with the interface device 200 and input information, receive, and decipher information using touch screen technology. Further, the interface device 200 can include an external device portal 204 wherein a manufacturer of the interface device 200 may place an external sensor. In many instances, the manufacturer of the interface device 200 may not deploy the additional sensor. The lack of a sensor may cause concern to patients and medical staff in that an empty space is seen when the interface device 200 is in operation without the additional sensor.

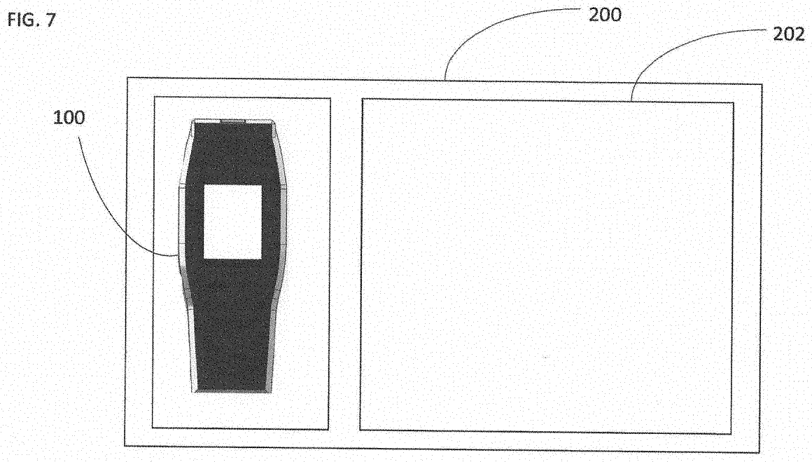

As best seen in FIG. 7, an embodiment of the system 100 can be seen embedded with an interface device 200. As shown, the existence of the system 100 occupying the empty space of the external device portal 204 mitigates patient and medical staff anxiety in that the empty external device portal is occupied and the system 100 is deployed as a support system for the interface device 200.

In some embodiments not shown, the system 100 can accompany a plurality of interface devices 200 configured to provide a plurality of monitoring services wherein accurate sensing of localized patient environmental conditions can provide enhanced monitoring and medical treatment efficiency.

In some embodiments, the systems, method or methods described above may be executed or carried out by a computing system including a tangible computer-readable storage medium, also described herein as a storage machine, that holds machine-readable instructions executable by a logic machine (i.e. a processor or programmable control device) to provide, implement, perform, and/or enact the above described methods, processes and/or tasks. When such methods and processes are implemented, the state of the storage machine may be changed to hold different data. For example, the storage machine may include memory devices such as various hard disk drives, CD, flash drives, SSD solid-state drive, cloud storage, or DVD devices. The logic machine may execute machine-readable instructions via one or more physical information and/or logic processing devices. For example, the logic machine may be configured to execute instructions to perform tasks for a computer program. The logic machine may include one or more processors to execute the machine-readable instructions. The computing system may include a display subsystem to display a graphical user interface (GUI) or any visual element of the methods or processes described above. For example, the display subsystem, storage machine, and logic machine may be integrated such that the above method may be executed while visual elements of the disclosed system and/or method are displayed on a display screen for user consumption. The computing system may include an input subsystem that receives user input. The input subsystem may be configured to connect to and receive input from devices such as a mouse, keyboard or gaming controller. For example, a user input may indicate a request that certain task is to be executed by the computing system, such as requesting the computing system to display any of the above described information, or requesting that the user input updates or modifies existing stored information for processing. A communication subsystem may allow the methods described above to be executed or provided over a computer network. For example, the communication subsystem may be configured to enable the computing system to communicate with a plurality of personal computing devices. The communication subsystem may include wired and/or wireless communication devices to facilitate networked communication. The described methods or processes may be executed, provided, or implemented for a user or one or more computing devices via a computer-program product such as via an application programming interface (API)

While the foregoing written description of the exemplary embodiments enables one of ordinary skill to make and use what is considered presently to be the best mode thereof, those of ordinary skill will understand and appreciate the existence of variations, combinations, and equivalents of the specific embodiment, method, and examples herein. The exemplary embodiments should therefore not be limited by the above described embodiment, method and examples, but all embodiments and methods within the scope and spirit of the exemplary embodiments as claimed.

Since many modifications, variations, and changes in detail can be made to the described preferred embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of the invention is much broader than presented by the claims. It is intended that broader claims will be submitted in an application that claims the benefit of priority from this application.

What is claimed is:

1. A medical environment monitoring system suitable for use for augmenting medical monitoring equipment for patients, the system comprising:
a handheld device configured to monitor environmental conditions, the device comprising:
at least one temperature sensor;
at least one humidity sensor; and
at least one chronometer, the chronometer structured to measure elapsed time; and
wherein the handheld device is contoured, shaped, and sized to occupy a cavity in the medical monitoring equipment such that when the handheld device is stowed onto the monitoring equipment and occupies the cavity, the cavity is not visible by plain sight; and
wherein the handheld device mates and connects with the medical diagnostic monitoring equipment in a releasable fashion.

2. The system in claim 1 further comprising a wireless communication module, the module configured to allow the medical environment monitoring system to communicate wirelessly with the medical monitoring equipment.

3. The system in claim 1 wherein the handheld device further comprises an anti-microbial coating, the coating designed to allow the handheld device to be utilized in medical sterile environments.

4. The system in claim 1 wherein the handheld device connects to the medical monitoring equipment with an articulating arm.

5. The system in claim 1 wherein the handheld device and the medical monitoring equipment are configured to provide real-time data transmission and reception and further designed to allow the medical monitoring equipment to process monitoring data that is calibrated with the data received from the handheld device.

6. A medical environment monitoring system suitable for use for augmenting medical monitoring equipment for patients, the system comprising:
a handheld device configured to monitor environmental conditions, the device comprising:
at least one temperature sensor;
at least one humidity sensor; and
at least one chronometer, the chronometer structured to measure elapsed time; and
wherein the handheld device is a standalone device.

7. The system in claim 6 further comprising a wireless communication module, the module configured to allow the medical environment monitoring system to communicate wirelessly with the medical monitoring equipment.

8. The system in claim 6 wherein the handheld device further comprises an anti-microbial coating, the coating designed to allow the handheld device to be utilized in medical sterile environments.

9. The system in claim 6 wherein the handheld device connects to the medical monitoring equipment with an articulating arm.

10. The system in claim 6 wherein the handheld device and the medical monitoring equipment are configured to provide real-time data transmission and reception and further designed to allow the medical monitoring equipment to process monitoring data that is calibrated with the data received from the handheld device.
